(19) United States
(12) Patent Application Publication
(10) Pub. No.: US 2005/0020958 A1
(43) Pub. Date: Jan. 27, 2005

(54) Control equipment and method for an extracorporeal blood circuit

(76) Inventors: Francesco Paolini, Ganaceto (IT); Francesco Fontanazzi, Modena (IT); Massimo Zaccarelli, San Felice sul Panaro (IT)

Correspondence Address: FINNEGAN, HENDERSON, FARABOW, GARRETT & DUNNER;LLP, 1300 I STREET, NW, WASHINGTON, DC, 20005, US

(21) Appl. No.: 10/500,324

(22) PCT Filed: Dec. 20, 2002
(86) PCT No.: PCT/IB02/05571

(30) Foreign Application Priority Data
Dec. 28, 2001 (IT) MI2001A002829

(51) Int. Cl. A61M 37/00; C02F 1/44; C02F 1/00

(52) U.S. Cl. 604/4.01; 210/739; 210/645

(57) ABSTRACT

Control equipment for an extracorporeal blood circuit (2), in which the extracorporeal circuit (2) has an access branch (8) and a return branch (10) connected to a blood treatment element (4; 4, 11a; 4, 11v; 25; 25, 11a; 25, 11v), is provided with a sensor for measuring a first temperature (TP) of the blood leaving a patient (P) along the access branch (8), with a heat exchanger formed by a portion (19) of the return branch (10) and by a device for regulating the temperature (T) of the blood coupled to the portion (19) of the return branch (10), and with a control unit (15) for operating the regulating device (18) as a function of the first temperature (TP) and of a reference temperature (Tset).

The present invention relates to control equipment for an extracorporeal blood circuit.

In particular, the present invention relates to control equipment for an extracorporeal blood circuit of a machine for purifying blood, to which the present invention will make specific reference without thereby relinquishing its general application.

The extracorporeal circuit is generally connected to the patient by means of an access needle and a return needle, which are inserted into a fistula formed in the patient's cardiovascular system, and are used, respectively, to collect the blood to be treated via an access branch, and to return the treated blood to the patient's cardiovascular system via a return branch.

A first known process for purifying the blood comprises, in addition to the extracorporeal circuit for the circulation of the blood, a circuit for the preparation of a treatment liquid or a circuit for the circulation of dialysate solutions which are ready for use and are commonly called “dialysate”, and a blood treatment element, which is commonly called a “filter”, and is divided into two compartments by a semi-permeable membrane.

One of the compartments of the filter, called the “blood compartment”, is connected to the extracorporeal circuit for the circulation of the blood and has the blood to be treated flowing through it during operation, while the other compartment of the filter has the dialysate flowing through it. The process of purifying the blood by means of a dialysate is called “haemodialysis”.

Another blood purification process, known as “haemofiltration”, is carried out by connecting the extracorporeal circuit to a filter, which is provided by a compartment through which the blood flows, and with a compartment acting as a receptacle for the undesired substances extracted from the blood.

A third process, which essentially combines the processes of haemodialysis and haemofiltration, is called haemodiafiltration.

During the blood purification treatment, the undesired particles contained in the blood migrate through the semi-permeable membrane from the blood compartment into the other compartment, either by convection (the phenomenon of convection is present in the process of haemofiltration, haemodialysis and haemodiafiltration), as a result of the passage of some of the blood liquid into the other compartment, or by diffusion (the phenomenon of diffusion is present in the processes of haemodialysis and haemodiafiltration), owing to the concentration gradient present between the blood and the dialysate.

Thus, at the end of the dialysis treatment, the patient will have lost some weight and the undesired substances will have been eliminated from the patient's blood.

The blood purification processes described above have variants which comprise the infusion of a replacement liquid into the extracorporeal circuit for the circulation of the blood, downstream of the filter (post-dilution) or upstream of the filter (pre-dilution).

In general, blood purification processes can be summarized as follows:
the pure haemofiltration process, where no treatment fluid is used;
the pre- or post-dilution haemofiltration process, where a replacement fluid is used upstream or downstream of the filter;
the haemodialysis process, where the dialysate is used alone; and
pre- or post-dilution haemodiafiltration processes, where both the dialysate liquid and the replacement liquid are used.

Given this general preliminary description, it should be noted that the blood extracted from the patient is normally at the temperature of 37° C. and is conveyed along the extracorporeal circuit for the circulation of blood to enable the purification treatment to be carried out. During its travel along the extracorporeal circuit, the blood undergoes temperature variations due to the heat exchange with the surrounding environment and with the treatment fluids, when the blood purification process makes use of a treatment fluid. A widespread practice, associated with the processes which make use of a blood treatment fluid, is that of heating the dialysate and/or the replacement liquid, to prevent the patient from being brought into a state of hypothermia. However, it is extremely difficult to predict what the thermal equilibrium of the blood will be in the extracorporeal circuit, in order to determine the exact amount of heat to be supplied to the blood via the dialysate and/or the replacement liquid, and thus to re-establish the initial blood temperature.

Moreover, a number of reliable studies have shown that the blood purification treatment frequently causes a rise in the patient's blood temperature, due to the specific reaction of the blood to the materials used, or in other words to the incomplete biocompatibility of these materials with the patient's blood.

In general, it is exceedingly difficult to implement in a dialysis machine a method capable of precisely determining the thermal equilibrium of the blood and of compensating the temperature variations to which the patient is subject. This is because, in order to implement such a method, it is necessary to determine the blood temperature in a precise way by means of temperature sensors of the non-invasive type, whose accuracy is sometimes relatively low, to determine in a precise way the rate of flow of blood in the extracorporeal circuit, to determine the temperature and rate of flow of the dialysate and/or of the replacement liquid (when the blood purification process makes use of a treatment fluid), and to determine various heat exchange coefficients. In practice, the thermal equilibrium of the blood in the extracorporeal circuit can be established in the laboratory by using highly sophisticated instruments, but is difficult to achieve in blood purification machines.

The patent EP 265,795 discloses blood control equipment applied to a blood purification machine. This equipment withdraws heat from the blood or supplies heat to it in the extracorporeal circuit for the circulation of blood, by suitably controlling the temperature of the dialysate and/or replacement liquid, and as a function of the difference between the temperature of the blood leaving the patient and a predetermined temperature, or as a function of the difference between the temperature of the blood leaving the patient and the temperature of the blood in the return branch, and also as a function of the rate of flow of the blood in the extracorporeal circuit.

The equipment described in EP 265,795 has numerous drawbacks, of which the following appear to bc most significant:
studies have demonstrated, as reported in the text of EP 265,795, that a low temperature of the dialysate promotes the attainment of greater stability of the cardiovascular system, and consequently of the pressure of the patient, and reduces the occurrence of feverishness in the patient. However, according to EP 265,795 the blood temperature is clearly controlled in an indirect way, by heating the replacement liquid and/or the dialysate;
the implementation of this control requires relatively complex equipment, and the drawing up of energy balances that are both accurate and complicated;
unless it is adapted, the equipment described in EP 265,795 cannot regulate the temperature in a machine providing treatment with dialysate and also in a machine operating with a replacement liquid;
the equipment cannot control the blood temperature in a machine providing a pure haemofiltration treatment.

The object of the present invention is to provide control equipment for an extracorporeal blood circuit which overcomes the drawbacks of the known control equipment and which, in particular, is both efficient and easily implemented in all blood purification machines.

According to the present invention, control equipment is provided for an extracorporeal blood circuit connected to a blood purification machine, in which the extracorporeal circuit comprises an access branch and a return branch connected to at least one blood treatment element; the equipment comprising a sensor for measuring a first temperature of the blood leaving a patient along the access branch upstream of the said blood treatment element, a control unit for regulating the blood temperature as a function of the first temperature and of a reference temperature; the equipment being characterized in that it comprises a device for regulating the blood temperature, connected to a portion of the return branch and downstream of the said blood treatment element.

The equipment according to the present invention makes it possible to dispense with the control of the temperature of the dialysate and/or replacement liquid. By suitably locating the regulation device within the return branch, it is possible to avoid the occurrence of phenomena which might further modify the blood temperature before the treated blood is returned to the patient. Furthermore, the control equipment interacts with the return branch and with the access branch only, and can be fitted to any blood purification machine.

The present invention also relates to a control method for an extracorporeal blood circuit.

According to the present invention, a control method is provided for an extracorporeal circuit for the circulation of blood in a blood purification machine, the extracorporeal circuit comprising an access branch and a return branch which are connected to at least one blood treatment element; the method comprising the steps of:
a) measuring a first temperature of the blood leaving a patient along the access branch; and
b) regulating the blood temperature as a function of the first temperature and of a reference temperature;
   the method being characterized in that the blood temperature is regulated along a portion of the return branch and downstream of the said blood treatment element.

To enable the present invention to be more clearly understood, a preferred embodiment thereof will now be described, purely by way of example and without restrictive intent, with reference to the attached figures, of which:

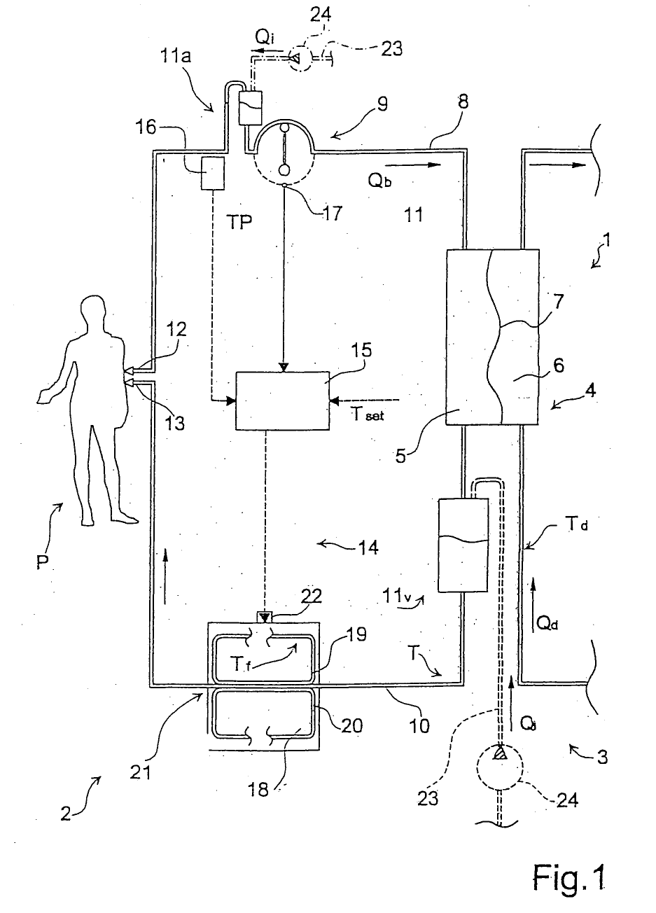

FIG. 1 is a schematic view, with parts removed for clarity, of a dialysis machine fitted with blood control equipment;

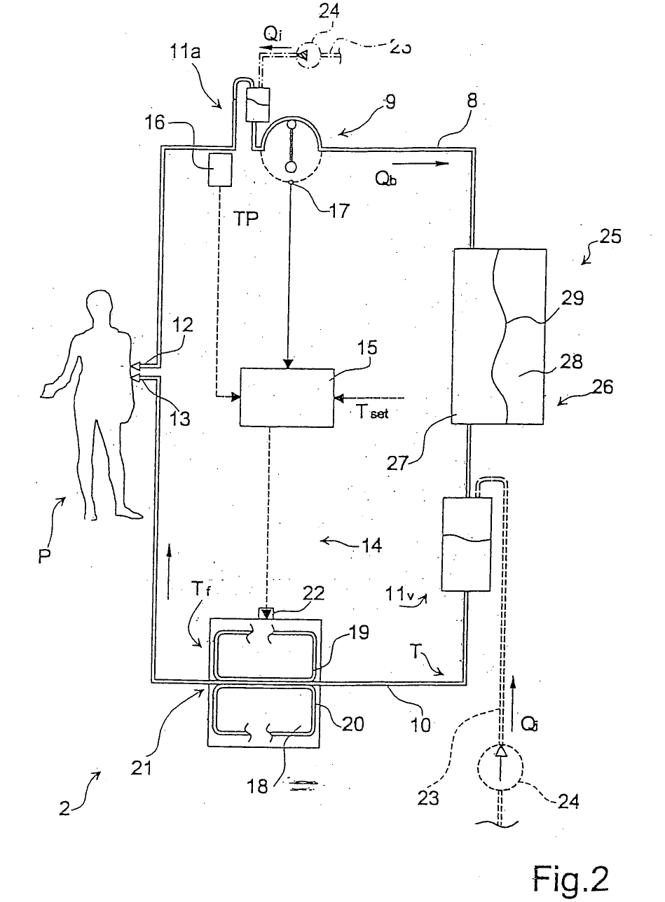

FIG. 2 is a schematic view of a haemofiltration machine fitted with the blood control equipment of FIG. 1.

In FIG. 1, the number 1 indicates the whole of a dialysis machine connected to a patient P. The machine 1 comprises an extracorporeal circuit 2 for the circulation of blood, a dialysate circuit 3 and a filter 4, which comprises a blood compartment 5 and a dialysate compartment 6 separated by a semi-permeable membrane 7.

The extracorporeal blood circuit 2 comprises an access branch 8, in which is located a peristaltic pump 9 providing a rate of blood flow Qb and an expansion chamber 11a upstream of the pump 9, and a return branch 10, in which an expansion chamber 11v is located. The access branch 8 has one end connected to the blood compartment 5 and one end provided with an access needle 12, which, during operation, is inserted into a fistula (not shown) in the patient P to collect the blood from the cardiovascular system of the patient P, while the return branch 10 has one end connected to the blood compartment 5 and an opposite end provided with a return needle 13, which, during operation, is inserted into the aforesaid fistula (not shown) to return the treated blood to the cardiovascular system of the patient P.

The machine 1 also comprises equipment 14 for regulating the blood temperature T in the extracorporeal circuit 2. The equipment 14 comprises a control unit 15 provided with a CPU, a temperature sensor 16 located in the access branch 8 upstream of the expansion chamber 11a, a sensor 17 to detect whether the peristaltic pump 9 is in operation, and a temperature regulator device 18 connected to a portion 19 of the return branch 10 downstream of the expansion chamber 11v, in such a way that it combines with the portion 19 to form a heat exchanger.

The device 18 regulates the blood temperature in the portion 19 without increasing the mass of the blood flow. In other words, the device 18 acts on a fluid which is physically separated from the blood and whose temperature Tf is controlled by the unit 15 in a range from 20° C. to 43° C., in such a way that heat is supplied to or withdrawn from the blood circulating in the return branch 10 directly before the blood is returned to the patient P.

The device 18 comprises at least one line 20 which forms a series of windings or a tube bundle, and provides a seat 21 for housing the portion 19 of the return branch 10, and a heater/cooler 22 connected to the control unit 15.

In operation, during the dialysis treatment the blood is collected from the patient P and is conveyed along the extracorporeal circuit 2 at the flow rate Qb, while the dialysate is conveyed along the circuit 3 at a flow rate Qd. The sensor 16 measures the temperature TP and the control unit 15 operates the device 18, according to a predetermined algorithm, as a function of the temperature TP and of a reference temperature Tset which is set by an operator in the control unit 15.

For example, the control unit 15 compares the temperature TP with a reference temperature Tset, which is generally equal to 37° C., and calculates the temperature difference AT between the temperature TP and the reference temperature Tset. At the start of the dialysis treatment the device 18 keeps the temperature Tf of the fluid at a value equal to the reference temperature Tset, while the temperature Td of the dialysate is regulated in such a way as to optimize the haemodialysis treatment. During the haemodialysis treatment the blood temperature T along the extracorporeal circuit 2 varies as a result of heat exchange with the surrounding environment, with the dialysate, and with the fluid conveyed within the device 18, and as a function of the reaction of the patient P to the materials used in the blood treatment.

The temperature TP is measured by the sensor 16, for example at relatively short intervals during the dialysis treatment, and the unit 15 calculates the temperature difference ΔT at the same frequency as that of the measurement of the temperature TP. When the temperature difference ΔT between the temperature TP and the reference temperature Tset takes a negative value, the temperature Tf of the fluid is raised in such a way as to supply heat to the blood along the portion 19, while when the temperature difference AT takes a positive value the temperature Tf of the fluid is lowered in such a way as to withdraw heat from the blood along the portion 19. By repeating the procedure described above at short intervals of time, it is possible to rapidly stabilize the temperature TP, in other words the temperature of the patient P, at a value close to the reference temperature Tset, whenever there is a variation of the temperature TP with respect to the reference temperature Tset.

The sensor 17 detects the state of operation of the pump 9 and emits a signal to indicate when the pump 9 is operational and when it is stopped. If the signal emitted by the sensor 17 indicates that the pump 9 is in a stopped state, the control unit 15 keeps the value of Tf equal to the reference temperature Tset; if, on the other hand, the signal indicates that the pump 9 is in an operational state, the fluid temperature Tf is regulated as a function of the temperature difference ΔT according to the procedure described above.

In a variant of the operation, the reference temperature Tset is not fixed, but varies during the dialysis treatment according to a specified profile.

In a variant, the machine 1 is equipped with an infusion line shown in broken lines in FIG. 1. The infusion line comprises an infusion branch 23 connected to the expansion chamber 11v of the return branch 10 and a pump 24 located in the branch 23 to provide a rate of flow Qi of replacement liquid which is introduced into the extracorporeal circuit 2. The replacement liquid can cause a further variation of the temperature T of the blood which is mixed with the replacement liquid.

The equipment 14 applied to the variant of FIG. 1, and its mode of operation, are completely identical to those described with reference to the circuit of FIG. 1 without the infusion process, although in the case of the variant the blood circulating in the extracorporeal circuit 2 is subjected to a first heat exchange in the blood compartment 5 of the filter 4 and to a second heat exchange in the expansion chamber 11v of the return branch 10. In this case, the beat generator 18 must be located downstream of the expansion chamber 11v of the return branch 10, to correct the variations of the blood temperature T before the blood is returned to the patient P.

In a further variant, the machine is equipped with an infusion line, which is shown in chained lines in FIG. 1, and comprises the infusion branch 23 connected to the expansion chamber 11a of the access branch 8 and the pump 24 for providing the rate of flow Qi of the infusion liquid. In this case also, both the equipment 14 and the operation of the equipment 14 remain unaltered with respect to the cases described previously.

With reference to FIG. 2, the number 25 indicates a haemofiltration machine, comprising the extracorporeal circuit 2 and a haemofiltration filter 26 comprising a blood compartment 27 and a compartment 28, separated by a semi-permeable membrane 29. The machine 25 is provided with blood control equipment 14, and also, in the variants illustrated in broken lines and in chained lines respectively, with a post-dilution and/or a pre-dilution infusion branch.

The machine 25 can carry out pure haemofiltration treatments and pre- and/or post-dilution haemofiltration treatments.

The equipment 14 applied to the machine 25, and its mode of operation, are completely identical to those associated with the machine 1.

The equipment 14 is particularly advantageous because it can be connected to any type of blood purification machine and does not require adaptation to the type of purification treatment which is administered.

1-25. (canceled)

26. An apparatus for control of an extracorporeal blood circuit connected to a blood purification machine, said extracorporeal blood circuit comprising an access branch connected to at least one blood treatment element and a return branch connected to said at least one blood treatment element; said apparatus comprising:
a sensor for measuring a first temperature of blood leaving a patient along the access branch upstream of said at least one blood treatment element;
a control unit for regulating a blood temperature in the extracorporeal blood circuit as a function of said first temperature and of a reference temperature; and
a regulating device for regulating said blood temperature in the extracorporeal blood circuit, connected to a portion of the return branch and downstream of said at least one blood treatment element.

27. An apparatus according to claim 26, wherein said regulating device is combined with said portion of the return branch to form a heat exchanger, said control unit being connected to said temperature regulating device.

28. An apparatus according to claim 26, wherein said regulating device comprises a line for conveying a fluid, said fluid being capable of being heated to a fluid temperature lying within a specified range about 37° C.

29. An apparatus according to claim 26, wherein said regulating device has a seat for housing said portion of the return branch.

30. An apparatus according to claim 26, wherein said extracorporeal blood circuit is connected to a pump for conveying blood along the extracorporeal blood circuit, and said regulating device comprises a line for conveying fluid, the apparatus comprising a sensor for detecting the operating state of said pump, the control unit maintaining the fluid temperature of said fluid equal to said reference temperature when said pump is not in operation.

31. An apparatus according to claim 26, wherein said return branch comprises an expansion chamber, said portion of the return branch being located downstream of the expansion chamber.

32. An apparatus according to claim 26, wherein said at least one blood treatment element is formed by a hemodialysis filter comprising a blood compartment and a dialysate compartment, said dialysate compartment having a dialysate flowing therein.

33. An apparatus according to claim 26, wherein said at least one blood treatment element comprises a hemodialysis filter comprising a blood compartment and a dialysate compartment, said dialysate compartment having a dialysate flowing therein, and an expansion chamber, said expansion chamber receiving a replacement fluid.

34. An apparatus according to claim 26, wherein said at least one blood treatment element is formed by a hemofiltration filter.

35. An apparatus according to claim 26, wherein said at least one blood treatment element comprises a hemofiltration filter and an expansion chamber, said expansion chamber receiving a replacement fluid.

36. An apparatus according to claim 26, wherein said control unit regulates the blood temperature in the extracorporeal blood circuit as a function of the first temperature and of the reference temperature at predetermined intervals of time.

37. An apparatus according to claim 26 or 36, wherein said control unit regulates the blood temperature in the extracorporeal blood circuit as a function of a difference between the first temperature and the reference temperature.

38. A control method for an extracorporeal blood circuit for the circulation of blood in a blood purification machine, the extracorporeal blood circuit comprising an access branch and a return branch, said access branch and return branch being connected to at least one blood treatment element; the control method comprising the steps of:
measuring a first temperature of blood leaving a patient along the access branch; and
regulating a blood temperature in the extracorporeal blood circuit as a function of the first temperature and of a reference temperature;
the blood temperature in the extracorporeal blood circuit being regulated along a portion of the return branch and downstream of said at least one blood treatment element.

39. A control method according to claim 38, wherein the steps of measuring a first temperature of blood leaving a patient along the access branch and of regulating the blood temperature in the extracorporeal blood circuit as a function of the first temperature and of a reference temperature are repeated at intervals of time.

40. A method according to claim 38, wherein a temperature difference between the first temperature and the reference temperature is calculated, said blood temperature in the extracorporeal blood circuit being regulated as a function of said temperature difference.

41. A method according to claim 40, further comprising a step of regulating a heat exchange of a heat exchanger, said heat exchanger comprising said portion of the return branch and a temperature regulating device connected to said portion of the return branch.

42. A method according to claim 40, wherein heat is withdrawn from blood along said portion of the return branch when said temperature difference is positive.

43. A method according to claim 40, wherein heat is supplied to the blood along said portion of the return branch when said temperature difference is negative.

44. A method according to claim 38, wherein a fluid is conveyed along said temperature regulating device, said fluid having a fluid temperature that varies within a specified range about 37° C.

45. A method according to claim 44, wherein blood is conveyed along the extracorporeal blood circuit by means of a pump, a state of operation of the pump being detected, the fluid temperature being regulated as a function of the first temperature and of the reference temperature, and the fluid temperature being kept equal to the reference temperature when the pump is not in operation.

46. A method according to claim 38, wherein the reference temperature is varied according to a specified profile.

47. A method according to claim 38, wherein said extracorporeal blood circuit is used for a hemodialysis treatment; said at least one blood treatment element being formed by a hemodialysis filter through which blood and a dialysate flow in a counterflow mode.

48. A method according claim 38, wherein said extracorporeal blood circuit is used for a hemodiafiltration treatment; said at least one blood treatment element comprising a hemodialysis filter through which blood and a dialysate flow in a counterflow mode, said at least one blood treatment element further comprising an expansion chamber supplied with a replacement fluid.

49. A method according to claim 38, wherein said extracorporeal blood circuit is used for a pure hemofiltration treatment, said at least one blood treatment element comprising a hemofiltration filter through which blood flows.

50. A method according to claim 38, wherein said extracorporeal blood circuit is used for a hemofiltration treatment, said at least one blood treatment element comprising a hemofiltration filter through which blood flows, said at least one blood treatment element further comprising an expansion chamber supplied with a replacement fluid.
